(19) United States
(12) Patent Application Publication
(10) Pub. No.: US 2026/0225971 A1
(43) Pub. Date: Aug. 6, 2026

(54) METHOD AND SYSTEM FOR CONVERTING A CARBONACEOUS FEEDSTOCK INTO H2 AND SOLID CARBON

(71) Applicant: BASE SE, Ludwigshafen am Rhein (DE)

(72) Inventors: Paul-Vinzent STROBEL, Ludwigshafen am Rhein (DE); Johannes BODE, Ludwigshafen am Rhein (DE); Jens Peter JOHANNSEN, Ludwigshafen am Rhein (DE); Andre BADER, Ludwigshafen am Rhein (DE); Dieter FLICK, Ludwigshafen am Rhein (DE)

(21) Appl. No.: 19/154,089

(22) PCT Filed: Jan. 29, 2024
(86) PCT No.: PCT/EP2024/052040

§ 371 Date: Aug. 6, 2025

(30) Foreign Application Priority Data
Feb. 7, 2023 (EP) 23155381.9

(51) Int. Cl. C07C 1/04; B01J 6/00; B01J 8/08; B01J 19/24; C01B 3/12; C01B 3/24; C01B 32/05; C10B 53/02; C10J 3/50; C10K 3/04

(52) U.S. Cl. CPC C07C 1/041; B01J 6/008; B01J 8/085; B01J 19/245; C01B 3/12; C01B 3/24; C01B 32/05; C10B 53/02; C10J 3/50; C10K 3/04; C01B 2203/0272; C01B 2203/0277; C01B 2203/0283; C01B 2203/1241; C10J 2300/0903; C10J 2300/0916; C10J 2300/0946; C10J 2300/0959; C10J 2300/1618; C10J 2300/1662

(57) ABSTRACT

The invention relates to a system and a method for converting a carbonaceous feedstock in a multistep procedure in which a) syngas is made from the carbonaceous feedstock, b) the syngas is converted by a methanation reaction into methane and c) methane is converted by a methane pyrolysis reaction into H₂ and sold carbon. Optionally, steps a) and b) can be carried out in a first location and the methane is transported to a second location where step c) is carried out.

### TECHNICAL AREA

The present invention relates to a system and a method for converting a carbonaceous feedstock into H₂ and solid carbon.

### BACKGROUND OF THE INVENTION

Hydrogen (H₂) produced with a low product carbon footprint (PCF) such as “green” H₂ is required as a feedstock for the (petro-)chemical industry and as an alternative fuel in the challenge to reduce emission of greenhouse gases such as CO₂. A major route to green H₂ is electrolysis of water utilizing electricity from renewable sources such as solar and wind energy. Another strategy for generation of H₂ having a low product carbon footprint (PCF) utilizes gasification of carbonaceous feedstocks such as waste wherein the feedstock (10) is partially oxidized in sub-stoichiometric amounts of O₂, air and/or steam in a gasification reaction (11) (FIG. 1). Such reactions form a gaseous product mixture (12) comprising syngas with varying ratios of H₂:carbon-oxides (CO and CO₂), impurities such as halides and sulfur-containing compounds and solid/highly viscous carbonaceous residues. Impurities are then removed from the gaseous product mixture (12) and a clean syngas is obtained.

The syngas composition and amount/type of impurities depend on the carbonaceous feedstock used and the process parameters applied during the gasification of the carbonaceous feedstock. The syngas has for example a molar ratio H₂:CO in the range of 0.5:1 to 2:1. The preferred carbonaceous feedstock is waste which is often a mixture of different components of varying amounts. Furthermore, such waste may also comprise different amounts of components which are referred to as biomass. For example, textiles can consist of cotton (=a biobased product) and a polymer made from feedstocks of fossil origin such as Nylon sometimes referred to as “synthetic fibers”.

The yield of H₂ may then be improved with a water-gas shift (WGS) reaction (14) whereby additional H₂ is formed by a full conversion of the CO present in the syngas with water to H₂ and CO₂. Hence, the amount of CO₂ in the product gas stream is increased in respect to the syngas (12) and the gaseous reaction products mainly consist of the desired H₂ (15) and the undesired greenhouse gas CO₂ (13).

The syngas obtained by gasification of a carbonaceous feedstock or the individual components H₂ or CO can then be used as a feedstock for manufacture of chemical products which, after use, become waste. Said waste can then be used as a carbonaceous feedstock for syngas production by gasification and a new cycle of chemical products can be manufactured from said syngas or the individual components H₂ or CO. A major drawback of such a recycling loop is the formation of the undesired greenhouse gas CO₂ during the gasification reaction and/or the water-gas shift (WGS) reaction.

Various methods are known in the art which are applied to avoid the undesired release of CO₂ into the atmosphere. The CO₂ can be removed from the product gas stream by carbon capture (CC) methods such as absorption and/or adsorption, depending on the partial pressure of the CO₂ in the product gas stream and the total pressure of the product gas stream. The ab-/adsorbed CO₂ may then be further subjected to storage (CCS) or utilization (CCU). However, CCS and CCU methods require for example expensive equipment and have high operational costs such as a high energy consumption which increase the product carbon footprint of the H₂.

Furthermore, carbonaceous feedstocks such as waste and/or biomass are usually collected and stored in other places than the locations where H₂ is utilized as a feedstock for the manufacture of one or more chemical products and/or as a fuel. Hence, there is often a mismatch of the location where the carbonaceous feedstock available and/or is stored, especially in case the carbonaceous feedstock is waste and/or biomass, and the location where the H₂ obtained from said waste is needed as a feedstock for the chemical industry and/or as a fuel. Pipeline networks (“grids”) for H₂ will not be ready in near future to overcome said mismatch of the location where the H₂ is generated from a carbonaceous feedstock such as waste and/or biomass and the location where the H₂ is required by the chemical industry as a feedstock for manufacture of chemical products and/or as a fuel.

US 2012/0241676 A1 discloses a method for gasification of a carbonaceous feedstock with reduced emission of CO₂. The method comprises gasification of the carbonaceous feedstock to form a gas containing CO, CO₂, methane, H₂O and H₂ followed by pyrolysis of the methane to solid C and H₂, and a reversed water-gas shift reaction to convert the CO₂ to CO. The final reaction product is syngas (H₂, CO) and solid C. This method is not useful as a route to H₂ having a low product carbon footprint (PCF) because considerable amounts of CO are formed. Furthermore, the whole method needs to be carried out in one location and either the carbonaceous feedstocks needs to be transported to the location where the H₂ is required as a feedstock for manufacture of chemical products and/or as a fuel or the H₂ has to be manufactured and transported from the location where the carbonaceous feedstock is available and/or stored to the location where the H₂ is used as a feedstock for manufacture of chemical products and/or as a fuel.

WO 2021/232158 A1 discloses a method for generating H₂ and a reduced amount of CO₂ by thermal cracking of a hydrocarbon. The use of solid and/or liquid carbonaceous feedstocks is not possible with a thermal cracking method utilizing the molten medium disclosed in this document. In addition, either the carbonaceous feedstock needs to be transported and stored in the location where the H₂ is used as a feedstock for manufacture of chemical products and/or as a fuel or the H₂ obtained by the carbonaceous feedstock needs to be transported to the location where the H₂ is used for manufacture of chemical products and/or as a fuel.

In addition, existing regulations and currently discussed future regulations such as the European Union (EU) “Carbon Removal Certification” framework aim to scale up carbon removal activities. The “Sustainable Carbon Cycles” (SCC) regulation requires an ongoing reduction of CO₂ emissions and even a net removal of CO₂ from the atmosphere. The methods described above produce CO₂ and/or CO which need a further treatment to prevent their emission into the atmosphere.

Based on the foregoing, there is a need for new systems and methods to produce H₂ from carbonaceous feedstocks such as waste and/or biomass while forming less or almost none of the greenhouse gas CO₂ and provide a solution for removal of the CO₂ from the atmosphere which overcome the disadvantages of known carbon capture (CC) methods. Furthermore, such new systems and methods should overcome the mismatch of the location where the carbonaceous feedstock such as waste is available and/or stored, and the location where the H₂ is required as a feedstock for manufacture of chemical products and other uses.

### SUMMARY OF THE INVENTION

These objectives are solved by a system for converting a carbonaceous feedstock into H₂ and solid carbon, the system comprising
at least one syngas producing unit,
a methanation unit and
a methane pyrolysis unit
wherein the methanation unit is downstream of and fluidically connected to the at least one syngas producing unit and
wherein said methane pyrolysis unit is downstream of and fluidically connected to said methanation unit
or wherein said methane pyrolysis unit is downstream of and connected by a natural gas pipeline grid to said methanation unit.

These objectives are further solved by a method for converting a carbonaceous feedstock into H₂ and solid carbon comprising, in this order, the steps
(i) providing a carbonaceous feedstock,
(ii) converting said carbonaceous feedstock into syngas,
(iii) removing impurities from said syngas to obtain a clean syngas, said clean syngas having a first molar ratio H₂:CO,
(iv) converting said clean syngas with a methanation reaction into methane and
(v) converting said methane with a methane pyrolysis reaction into H₂ and solid carbon.

The carbonaceous feedstocks are converted into raw syngas and solid/highly viscous carbonaceous residues such as ash and/or tar in at least one syngas producing unit such as a gasification island comprising at least one gasifier. Said solid/highly viscous carbonaceous residues can either get disposed as solid waste or subjected to other uses such as incineration or as a feedstock in a secondary reformer/gasifier to obtain further syngas.

Impurities are removed from the raw syngas and the clean syngas obtained is then converted in a methanation reaction to methane and water. Said methane is then converted to H₂ and solid carbon with a methane pyrolysis reaction in a methane pyrolysis unit which is downstream of the methanation unit.

In one embodiment of the present invention, the methane pyrolysis unit is downstream of and fluidically connected to the methanation unit. In this embodiment of the present invention, the methanation unit and the methane pyrolysis unit are installed in the same location.

In another embodiment of the present invention, the methane pyrolysis unit is downstream of and connected by a natural gas pipeline grid to the methanation unit. In this embodiment of the present invention, the methanation unit and the methane pyrolysis unit are installed in different locations.

Preferably, steps (i) to (iv) are carried out in a first location where the carbonaceous feedstock such as for example waste and/or biomass is available and/or stored and the methane obtained in step (iv) is then inserted into a suitable pipeline such as a natural gas pipeline grid and transferred in said pipeline to a second location where the methane obtained in step (iv) is taken from the natural gas pipeline grid and transported to a methane pyrolysis reaction unit installed in said second location to convert said methane with a methane pyrolysis reaction into H₂ and solid carbon (step (v)). Said second location where the methane pyrolysis unit is installed is preferably a location where H₂ is required as a feedstock for manufacturing of at least one chemical product and/or where the H₂ can be utilized as a fuel.

The methane obtained in step (iv) can be taken from the natural gas pipeline grid and immediately fed into the methane pyrolysis unit or said methane can be transferred from the natural gas pipeline grid to a means for storage and later transferred from said means for storage to the methane pyrolysis unit. The means for storage can be for example a tank suitable for storage of methane such as storage tank or the natural gas pipeline grid itself.

This embodiment of the present invention is a method for converting a carbonaceous feedstock into H₂ and solid carbon wherein the methane formed in step (iv) is transported through a natural gas pipeline grid in an additional step (iv′) prior to converting the methane with a methane pyrolysis reaction into H₂ and solid carbon in step (v) and wherein steps (i) to (iv) are carried out in a first location and step (v) is carried out in a second location and wherein the first location and the second location are different from each other.

Accordingly, in this embodiment of the present invention the methane pyrolysis unit is also downstream of the methanation unit but the methane pyrolysis unit is not directly fluidically connected to said methanation unit because the methanation unit and the methane pyrolysis unit are operating in different locations and are separated by a natural gas pipeline grid.

The solid carbon formed during the methane pyrolysis reaction can be used in various ways: for example, as fresh catalyst material for the methane pyrolysis reaction, in aluminum and steel production, in tire manufacturing, electrode manufacturing, polymer blending, as additive for construction materials, in devices made of or comprising carbon such as heat exchangers, in soil conditioning or for renaturation of former opencast lignite mining sites.

The CO₂ formed in the system and the method according to the present invention is removed from the atmosphere by formation of solid carbon in the methane pyrolysis unit and step (v).

The H₂ formed can be utilized as an energy source or a feedstock for synthesis of one or more chemical products such as for example methanol and olefins via methanol-to-olefin (MTO) synthesis.

In case of a preferred embodiment of the present invention, the H₂ is formed at the location (second location) where it is needed from methane, the methane formed at the location where the carbonaceous feedstock such as waste and/or biomass is available and/or stored (first location). The methane can be transported through existing natural gas pipeline grid or, if required, through new pipeline grids. Pipeline gids for transport of natural gas are technically less complex and, accordingly, require less capital investment for construction and have less operational costs than pipeline grids dedicated for the transport of H₂.

The amount of CO₂ disposed during the method according to the present invention is reduced in respect to methods for H₂ manufacture from carbonaceous feedstock known in the art while at the same time the amount of H₂ formed is comparable or even higher. Moreover, a considerable portion of the carbon present in the carbonaceous feedstock is completely removed from the atmosphere by conversion into solid carbon.

In further embodiments of the present invention none or almost none of the carbon present in the carbonaceous feedstock is released to the atmosphere in form of CO₂ and/or CO but converted into solid carbon.

### DESCRIPTION OF THE FIGURES

FIG. 1 shows a system and method for converting a carbonaceous feedstock into H₂ according to prior art.

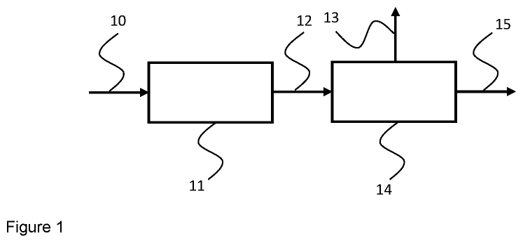

FIG. 2 shows the system and method for converting a carbonaceous feedstock into H₂ and solid carbon according to a first embodiment of the present invention.

FIG. 3 shows the system and method for converting a carbonaceous feedstock into H₂ and solid carbon according to a second embodiment of the present invention.

FIG. 4 shows the system and method for converting a carbonaceous feedstock into H₂ and solid carbon according to a third embodiment of the present invention.

FIG. 5 shows the system and method for converting a carbonaceous feedstock into H₂ and solid carbon according to a fourth embodiment of the present invention.

FIG. 6 shows the system and method for converting a carbonaceous feedstock into H₂ and solid carbon according to a fifth embodiment of the present invention.

FIG. 7 shows the system and method for converting a carbonaceous feedstock into H₂ and solid carbon as part of a closed recycling loop.

### DETAILED DESCRIPTION OF THE INVENTION

The present invention is further described below with reference to the embodiments and figures, but the present invention is not limited to these embodiments, and any modifications or substitutions within the basic spirit of the present invention are still within the scope of the present invention as claimed.

### Definitions

The term “syngas” refers to a mixture of H₂ and CO in various molar ratios. “Syngas” may comprise further constituents such as CO₂ and remaining impurities after purification.

The term “syngas producing unit” refers to a production facility in which syngas is obtained from a carbonaceous feedstock. Such a “syngas producing unit” comprises at least one “pretreatment unit”, at least one “syngas producing reactor unit” such as a gasifier wherein the syngas is formed by a chemical reaction and at least one “syngas purification unit” in which impurities present in the raw syngas formed in the at least one syngas producing reactor unit are removed. A carbonaceous feedstock is pre-treated in a “pre-treatment unit” which includes one or more of physical, mechanical and/or chemical pre-treatment procedures. In the most simple case, the carbonaceous feedstock is just transferred in a “pre-treatment unit” (=simplest form of mechanical pre-treatment) into the “syngas producing reactor unit”.

The term “syngas upgrading unit” refers to at least one process unit in which the H₂ content of syngas is increased to obtain a higher molar ratio H₂:CO. Examples for a “syngas upgrading unit” are water-gas shift units and H₂ injection units.

The term “upstream of” is defined herein in respect to a succession of unit operations as located next to on the side which is against the flow direction of fluids passing said succession of unit operations.

The term “downstream of” is defined herein in respect to a succession of unit operations as located next to on the side which is in the flow direction of fluids passing said succession of unit operations.

The term “fluidically connected to” in respect to two or more units such as a syngas producing unit and a methanation unit is defined herein that a fluid such as solids, liquids, gases and mixtures of the aforementioned can flow from one of such unit to the other such unit. Two units “fluidically connected to” each other are for example connected by one or more pipes which each other or by screw conveyors or by extruders or by solids pumps.

A pipeline grid such as a natural gas pipeline grid which can transport methane leaving a methanation unit placed in a first location to a methane pyrolysis unit placed in a second location is not suitable to constitute a “fluidically connected to” link between two units such as said methanation unit and said methane pyrolysis unit because said pipeline separates said units by kilometers or tens of kilometers or hundreds of kilometers or even thousands of kilometers. Nevertheless, also in case the methanation unit and the methane pyrolysis unit are separated by such a distance, the methane pyrolysis unit is considered “downstream of” the methanation unit and “connected to” said methanation unit because the methane formed in the methanation unit leaves the methanation unit and enters the methane pyrolysis unit after transportation in a natural gas pipeline grid and an optional storage of the methane in between the methanation unit and the methane pyrolysis unit.

The term “natural gas pipeline grid” is defined herein as pipelines or a network of pipelines used to transport natural gas and methane or which are/is suitable for the transport of natural gas and methane. “Natural gas pipeline grids” are also known as “natural gas networks”.

The objectives described above are solved by a system for converting a carbonaceous feedstock into H₂ and solid carbon according to a first embodiment of the present invention, the system comprising
at least one syngas producing unit,
a methanation unit,
a methane pyrolysis unit and
wherein the methanation producing unit is downstream of and fluidically connected to said at least one syngas producing unit and wherein said methane pyrolysis unit is downstream of and fluidically connected to said methanation unit or said methane pyrolysis unit is downstream of and connected to the methanation unit by a natural gas pipeline grid.

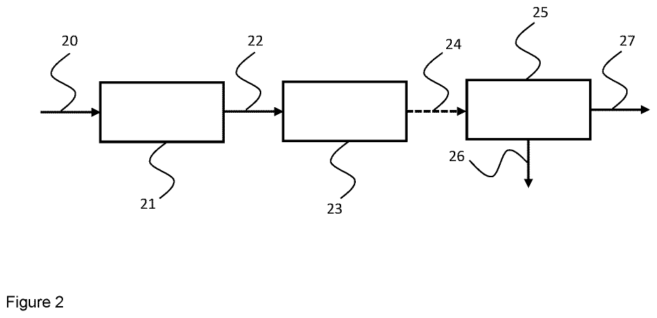

The system according to this first embodiment of the present invention is further explained with FIG. 2: a carbonaceous feedstock (20) is introduced into at least one syngas producing unit (21) where said carbonaceous feedstock is converted into raw syngas. Said raw syngas is then treated in at least one syngas purification unit which is part of the at least one syngas producing unit (21) and leaves the at least one syngas producing unit (21) as a clean syngas (22) having a first molar ratio H₂:CO. Next, said clean syngas (22) enters a methanation unit (23). The methanation unit (23) is downstream of and fluidically connected to the syngas producing unit (21). The clean syngas (22) is converted in the methanation unit (23) into methane (24), more particularly into a product stream comprising methane and water. The at least one syngas producing unit (21) and the methanation unit (23) are preferably placed in a first location where the carbonaceous feedstock (20) is available and/or stored.

In one embodiment of the present invention, the methane (24) leaves the methanation unit (23) and is then transferred to a pipeline grid which is dedicated to the transport of natural gas and methane (“natural gas pipeline grid”). The methane (24) is transported in said natural gas pipeline grid to a second location where H₂ is required as a feedstock for production of at least one chemical product and/or as a fuel, leaves at the second location said natural gas pipeline grid and is transferred to a methane pyrolysis unit (25) installed in the second location or temporarily stored in a means for storing the methane (24) such as a tank or the natural gas pipeline grid. Hence, the methane pyrolysis unit (25) is downstream of the methanation unit (23) but not fluidically connected to the methanation unit (23) because the methane pyrolysis unit (25) is placed in a second location which is different from said first location where the at least one syngas producing unit (21) and the methanation unit (23) are installed. Methane (24) is then converted in the methane pyrolysis unit (25) into a stream of H₂ (27) and solid carbon (26) which both leave the methane pyrolysis reactor (25).

In another embodiment of the present invention, the methane (24) leaves the methanation unit (23) and enters the methane pyrolysis unit (25) which is also installed in said first location where also the at least one syngas producing unit (21) and the methanation unit (23) are installed and the carbonaceous feedstock (20) is available and/or stored. The methane pyrolysis unit (25) is downstream of and fluidically connected to the methanation unit (23) in this embodiment of the present invention. Methane (24) is converted in the methane pyrolysis unit (25) into a stream of H₂ (27) and solid carbon (26) which both leave the methane pyrolysis reactor (25).

The syngas producing unit (21) comprises at least one syngas producing reactor which is downstream of and fluidically connected to at least one pre-treatment unit. The syngas producing unit (21) further comprises at least one syngas purification unit which is downstream of and fluidically connected to the at least one syngas producing reactor. The at least one syngas producing reactor unit in a syngas producing unit (21) can be any reactor or combination of units which is/are suitable to convert a carbonaceous feedstock into raw syngas. The at least one syngas producing reactor unit can be for example a gasifier in which a carbonaceous feedstock is converted into raw syngas. The at least one syngas producing unit (21) further comprises at least one syngas purification unit for removing impurities in the raw syngas made in the at least one syngas producing reactor unit to obtain a clean syngas (22) from the at least one syngas producing unit (21), said clean syngas (22) having a first molar ratio H₂:CO.

Preferably, the at least one syngas producing unit (21) comprises at least one gasifier as syngas producing reactor unit. Most preferably, the at least one syngas producing unit is a gasification island comprising at least one gasifier as syngas producing reactor unit.

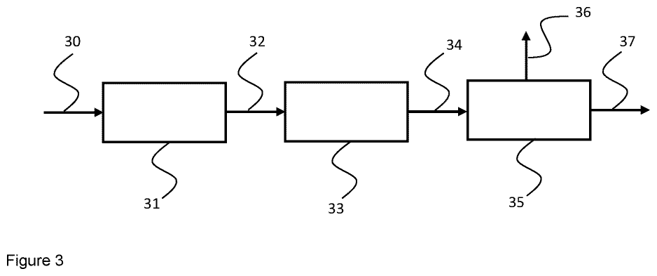

A system wherein the at least one syngas producing unit is a gasification island comprising at least one gasifier is the second embodiment of the present invention. A gasification island suitable as syngas producing unit (21) is shown in FIG. 3.

The gasification island comprises at least one pre-treatment unit (31) for a carbonaceous feedstock (30) which pre-treat and/or transport and/or provide the carbonaceous feedstock to the at least one gasifier (33). A gasification island further comprises at least one syngas purification unit (35) in which impurities (36) in the raw syngas (34) formed by the gasification reaction are removed to obtain a clean syngas (37) having a first molar ratio H₂:CO.

In a gasification island the at least one gasifier (33) is downstream of and fluidically connected to the at least one pre-treatment unit (31). The at least one syngas purification unit (35) is downstream of and fluidically connected to the at least one gasifier (33).

Suitable gasifiers (31) comprise counter-current fixed bed reactors, co-current-fixed bed reactors, bubbling fluidized bed reactors, circulation fluidized bed reactors, and downdraft or updraft entrained flow reactors. The selection of size and reactor type depends on several parameters, including the composition of the carbonaceous feedstock, demand of products, moisture content and availability of the carbonaceous feedstock. Preferably, the gasifier (31) is an “oxygen blown” gasifier, i.e., oxygen is preferably used as the oxidant in suitable gasifiers (31) listed above.

The carbonaceous feedstock (20;30) is preferably a solid and/or liquid material or mixture of materials which comprises organic compounds and/or organic polymers. Said organic compounds and/or organic polymers contain biobased (¹⁴C) carbon and/or carbon of fossil sources and/or carbon which has been at least once recycled (“recycle content carbon”). The carbonaceous feedstock may further contain impurities such as inorganic components and metallic components. Preferably, the carbonaceous feedstock is a solid and/or liquid carbonaceous feedstock and is selected from the group comprising (raw) biomass, biomass derived liquids, waste and mixtures thereof, and mixtures of the aforementioned with fossil feedstocks such as coal, oil and natural gas.

The term “biomass” includes but is not limited to wood, wood pellets, wood chips, straw, lignocellulosic biomass, energy crops, algae, agricultural residues such as switch grass, discarded seed corn, corn stover and other crop residues.

Raw biomass can be upgraded into better and more practical form by compacting (e.g., wood is compacted to wood pellets) or by different conversions classified as thermal (e.g., torrefaction and/or pyrolysis), chemical, and biological.

The term “biomass derived liquids” comprises pyrolysis oil and biomass derived fuels.

The term “waste” comprises municipal solid waste (MSW), textiles, industrial waste, wood processing residues, waste wood, logging residues, agricultural/farming residues, sewage sludge, plastic waste, shredder residues such as car shredder residues (“automotive shredder residues”) and mixtures of the aforementioned.

Preferably, the carbonaceous feedstock (20;30) is selected from the group comprising biomass, municipal solid waste (MSW), shredder residues such as car shredder residues, textiles and mixtures of the aforementioned.

Preferably, the carbonaceous feedstock is converted into syngas by a gasification reaction and/or the carbonaceous feedstock is selected from the group comprising selected from the group comprising biomass, municipal solid waste (MSW), shredder residues such as car shredder residues, textiles and mixtures of the aforementioned.

The system according to the present invention is suitable for a method for converting a carbonaceous feedstock into H₂ and solid carbon, said method comprising the steps
(i) providing a solid and/or liquid carbonaceous feedstock,
(ii) converting said solid and/or liquid carbonaceous feedstock into a raw syngas,
(iii) removing impurities from said syngas to obtain a clean syngas, said clean syngas having a first molar ratio H₂:CO,
(iv) converting said clean syngas with a methanation reaction into methane and
(v) converting said methane with a methane pyrolysis reaction into H₂ and solid carbon.

The system according to the present invention is also suitable for the following method: Subjecting a methane stream to methane pyrolysis, wherein the methane stream is derived from a process comprising
(i) conversion of a solid and/or liquid carbonaceous feedstock, preferably by gasification to obtain raw syngas,
(ii) removing impurities from said syngas to obtain a clean syngas, said clean syngas having a first molar ratio H₂:CO and
(iii) methanation of said clean syngas to obtain said methane stream.

This method is shown in FIG. 2. A carbonaceous feedstock (20) is provided. Said carbonaceous feedstock is optionally subjected to one or more pre-treatment steps before converting said solid and/or liquid carbonaceous feedstock into a raw syngas such as sorting, separation, mechanical size reduction, biological treatment, drying, torrefaction, grinding, milling, agglomeration and pelletizing. The suitable pre-treatment step(s) depend on the type of carbonaceous feedstock and on the type of syngas producing reactor, which is preferably a gasifier. The skilled person knows how to choose suitable pre-treatment step(s). Such optional pre-treatment step(s) can improve the conversion of the carbonaceous feedstock into raw syngas. Said conversion is preferably by a gasification reaction.

Next, the carbonaceous feedstock is converted into a raw syngas in at least one syngas producing unit (21), preferably by a gasification reaction in at least one gasifier, most preferably using a gasification island comprising at least one gasifier. A gasification reaction usually results in further reaction products such as solid and/or highly viscous carbonaceous residues (e.g., char and/or tar) which are further treated in separate steps not relevant for the method according to the present invention. Such “further reaction products” are different from the impurities present in raw syngas and, accordingly, don't need to be removed in a syngas purification unit. Such “further reaction products” can be for example separated in a cooling trap and then be recycled into the hot zone of the gasifier or a second gasifier which is preferably an entrained gasifier.

The gasification reaction in a gasifier is typically carried out at a temperature>700° C. in the presence of a sub-stoichiometric amount of an oxidant such as oxygen, air, steam, supercritical water or a mixture of the aforementioned. Oxygen is the most common oxidant used for gasification because of its easy availability and low cost. If steam acts as oxidant, the raw syngas has a higher first molar ratio H₂:CO than in case if oxygen is used as oxidant. For example, a the molar ratio carbonaceous feedstock:oxygen can range from 1.5:1 to 1.8:1.

The conversion of a carbonaceous feedstock in the syngas producing unit which preferably is a gasification island comprising at least one gasifier produces a raw syngas which consists primarily of H₂, CO, CO₂, methane, other hydrocarbons and impurities. Said raw syngas has a dedicated molar ratio H₂:CO when leaving the gasifier which ranges from about 0.1:1 to about 2:1 and depends on the type of solid and/or liquid carbonaceous feedstock used, the oxidant and other reaction conditions applied such as temperature and/or residence time for the gasification reaction.

Impurities in said raw syngas are removed from the raw syngas product stream directly after leaving the syngas producing reactor, preferably the gasifier in at least one syngas purification unit. In a preferred embodiment of the present invention, the raw syngas is obtained from the carbonaceous feedstock and purified to obtain a clean syngas having a first molar ratio H₂:CO in a gasification island (FIG. 3) comprising at least one pre-treatment units (31), at least one gasifier (33) which is downstream of and fluidically connected to said at least one pre-treatment unit, and at least one syngas purification unit (35) downstream of and fluidically connected to the at least one gasifier (33).

The carbonaceous feedstock (30) enters said at least one pre-treatment unit (31), the pretreated carbonaceous feedstock (32) enters the at least one gasifier (33) where it is converted into raw syngas (34). Said raw syngas (34) is then purified in the at least one syngas purification unit (35) and leaves said at least one syngas purification units (35) as a clean syngas (37).

Other gaseous substances such as HCl and H₂S are formed and/or separated from the raw syngas (34) in the at least one syngas purification unit (35). The impurities (36) are removed from the syngas and the clean syngas (37) having a first molar ration H₂:CO then enters the methanation unit (not shown in FIG. 3) where it is converted into methane and the methane is then converted into H₂ and solid carbon in a methane pyrolysis unit (not shown in FIG. 3).

The use of a clean syngas (22;37) obtained from the at least one syngas purification unit is preferred because catalysts utilized in successive process steps have an improved lifetime and maintain their activity when using a clean syngas (22;37) instead of the raw syngas obtained directly from the gasification reaction in the at least one gasifier.

Typical impurities in the raw syngas obtained from the gasification reaction in a gasifier comprise chlorides, sulfur-containing organic compounds such as sulfur dioxide, trace heavy metals (e.g., as respective salts) and particulate residues. Various chemical and/or physical methods for removal of such impurities from said raw syngas such as filtration, scrubbing, hydrotreatment and ab-/adsorption are known and can be chosen and adapted according to the type and respective concentration of the impurities in said raw syngas and the tolerance to such impurities in the successive process steps. Some selected methods for removal of impurities from said raw syngas will be discussed in more detail. One or more of said methods can also be implemented into the at least one syngas purification unit (35) of a gasification island (FIG. 3). However, this selection of methods is not limiting the scope of the present invention.

Bulk particulate impurities can be removed from the raw syngas by a cyclone and/or filters, fine particles and chlorides by wet scrubbing, trace heavy metals, catalytic hydrolysis for converting sulfur-containing organic compounds to H₂S and acid gas removal for extracting sulfur-containing gases such as H₂S. Bulky and fine particles in the syngas may also be removed with a quench in a soot water washing unit.

Next, the clean syngas (22;37) having a first molar ratio H₂:CO is subjected to a methanation reaction in a methanation unit (23) resulting in a gaseous product stream (24) comprising methane. The methanation unit (23) is downstream of and fluidically connected to the at least one syngas producing unit (21) and/or the at least one syngas purification unit (35) of the gasification island.

The methanation reaction is described by chemical reaction schemes (1) and (2):

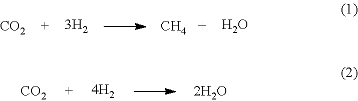

The methanation reaction (step (iv)) and suitable methanation units (23) are for example described in S. Rönsch, J. Schneider, S. Matthischke, M. Schlüter, M. Götz, J. Lefebvre, P. Prabhakaran, S. Bajohr: Review on methanation—From fundamentals to current projects; Fuel 166 (2016) 276-296 and can be selected and adapted by the skilled person.

The methanation reaction (step (iv)) is for example a catalytic reaction using nickel on alumina catalysts, preferably a honeycomb shape catalyst, at 1 to 70 bar and 200 to 700° C., preferably 5 to 60 bar, more preferably 10 to 45 bar and preferably 200 to 550° C., more preferably 10 to 45 bar.

Next, the methane (24) obtained in the methanation unit (23) is used as the feedstock for a methane pyrolysis reaction in a methane pyrolysis unit (25).

In one embodiment of the present invention, the methane pyrolysis unit (25) is downstream of and fluidically connected to the methanation unit (23). Accordingly, in this embodiment of the present invention the at least one syngas producing unit (21), the methanation unit (23) and the methane pyrolysis unit (25) are all installed in the same location.

In another embodiment of the present invention, the at least one syngas producing unit (21) and the methanation unit (23) are installed in the same location (“first location”) and the methane pyrolysis unit (25) is downstream of the methanation unit (23) but not fluidically connected to said methanation unit (23). Instead, the methane (24) formed in the methanation unit (23) is transferred to a natural gas pipeline grid and transported in the natural gas pipeline grid to another location (“second location”) and then either inserted to a methane pyrolysis unit (25) or temporarily stored in a means for storage and later inserted into a methane pyrolysis unit (25) in which the methane is converted into H₂ (27) and solid carbon (26). The first location and the second location are different from each other. The distance between the first location (methane producing location) and the second location (methane consumption location) is at least one kilometer or at least ten kilometer or at least hundred kilometer or even thousand or more kilometer.

The advantage of having the at least one syngas producing unit (21) and the methanation unit (23) installed in a first location and the methane pyrolysis unit (25) in a second location are:
methane is produced from the carbonaceous feedstock (20;32) at or near the location where the carbonaceous feedstock (20;32) is available and/or where sufficient space is available to store said carbonaceous feedstock (20;32) prior to use as a feedstock,
the available natural gas pipeline grid can be used to transport methane (24) from a first location to a second location,
H₂ is produced at a second location where it is required and/or needed as a feedstock for the manufacture of at least one chemical product and/or as a fuel.

The product gas stream comprising methane (24) leaving the methanation unit (23) is then converted to a product gas stream comprising H₂ (27) and solid carbon (26) by a methane pyrolysis reaction in a methane pyrolysis unit (25). The methane pyrolysis reaction is described by chemical reaction scheme (3):

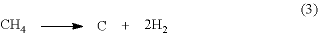

Optionally, the methane (24) obtained from the methanation unit (23) is first subjected to a purification and conditioning feeding the methane (24) into the methane pyrolysis unit (25). Technology for purification and conditioning of the gaseous products from the methanation is well known in the art, e.g., U.S. Pat. No. 8,568,512 and F. G. Kerry: Industrial Gas Handbook: Gas Separation and Purification. Typically, the following processes are used for methane purification: amine washing, pressurized water washing, pressure swing adsorption, physical adsorption, membrane processes and cryogenic processes. The second product water would be purified using standard methods in chemical engineering as well like extraction, membrane processes, adsorption and ion exchange.

The methane pyrolysis in the system and method according to the present invention can be conducted in different ways known to the persons skilled in the art: catalytically or thermally, and with heat input via plasma, resistance heating, liquid metal processes or autothermal (see for example N. Muradov and T. Veziroglu: “Green” path from fossil-based to hydrogen economy: An overview of carbon-neutral technologies”, International Journal Hydrogen Energy 33 (2008) 6804-6839, H. F. Abbas and W. M. A. Wan Daud: Hydrogen production by methane decomposition: A review, International Journal Hydrogen Energy 35 (2010) 1160-1190), R. Dagle et al.: An Overview of Natural Gas Conversion Technologies for Co-Production of Hydrogen and Value-Added Solid Carbon Products, Report by Argonne National Laboratory and Pacific Northwest National Laboratory (ANL-17/11, PNNL-26726) November 2017).

In case of autothermal methane pyrolysis, oxygen is introduced into the reaction for a partial oxidation of methane and H₂ for heat generation. In this case, the reactor effluent will become a syngas and contain CO and CO₂. This gas can be used internally or externally of the systems and methods according to the present invention. In one embodiment of the present invention, syngas obtained by said partial oxidation is combined with the clean syngas having a first molar ratio H₂:CO and/or a second syngas having a second molar ratio H₂:CO. Said “combined” syngas may be used for the synthesis of e.g., methanol.

The methane pyrolysis reactor may operate at 500 to 2000° C. dependent on the presence of any catalyst (preferably 500 to 1000° C.) or without a catalyst (preferably 1000 to 2000° C.). The thermal decomposition reaction is preferably conducted in a pressure range from atmospheric pressure to 30 bar.

If needed, additional methane from an external source can be fed into the reactor of the methane pyrolysis. Methane comprising biobased (¹⁴C) carbon such as biomethane is a preferred external source.

The solid carbon (26) type generated in the methane pyrolysis in the methane pyrolysis unit (25) depends on the reaction conditions, reactor and heating technology. Example products are
carbon black from plasma processes
carbon powder from liquid metal processes
granular carbon from thermal decomposition in fixed, moving or fluidized bed reactors.

Applications for solid carbon (26) obtained from methane pyrolysis in the methane pyrolysis unit (25) are discussed e.g., for aluminum and steel production, tire manufacturing, electrode manufacturing, polymer blending, additive for construction materials, carbon devices like heat exchangers, soil conditioning, or even storage.

The system is also according to the present invention is also suitable for a method for converting a carbonaceous feedstock into H₂ and solid carbon according to any of claims 8 to 14 wherein the methane formed in step (iv) is transported through a natural gas pipeline grid in an additional step (iv′) prior to converting the methane with a methane pyrolysis reaction into H₂ and solid carbon in step (v) and wherein steps (i) to (iv) are carried out in a first location and step (v) is carried out in a second location and wherein the first location and the second location are different from each other.

The system is also according to the present invention is also suitable for subjecting a methane stream to methane pyrolysis and obtaining H₂ and solid carbon from said methane pyrolysis, wherein the methane stream is derived from a process comprising
(i) gasification of a solid and/or liquid carbonaceous feedstock which comprises a chemical product to obtain syngas,
(ii) removing impurities from said syngas to obtain a clean syngas, said clean syngas having a first molar ratio H₂:CO and
(iii) methanation of said clean syngas to obtain the methane stream
wherein said chemical product, comprised in the said carbonaceous feedstock, was formed directly or indirectly from said H₂ obtained by the methane pyrolysis reaction and at least one second feedstock containing one or more carbon atom.

The system is also according to the present invention is also suitable for a use of a methane stream for methane pyrolysis, wherein the methane stream is derived from a method comprising the steps
(i) gasification of a solid and/or liquid carbonaceous feedstock to obtain syngas,
(ii) removing impurities from said syngas to obtain a clean syngas, said clean syngas having a first molar ratio H₂:CO and
(iii) methanation of said clean syngas to obtain the methane stream.

The systems and methods according to the first and second embodiments of the present invention have several advantages compared to methods disclosed in prior art:
an increased amount of carbon present in the carbonaceous feedstock is converted to solid carbon which can be further utilized,
H₂ is formed with a low product carbon footprint (PCF), potentially with negative PCF,
less or none of the greenhouse gas CO₂ is emitted to the atmosphere.

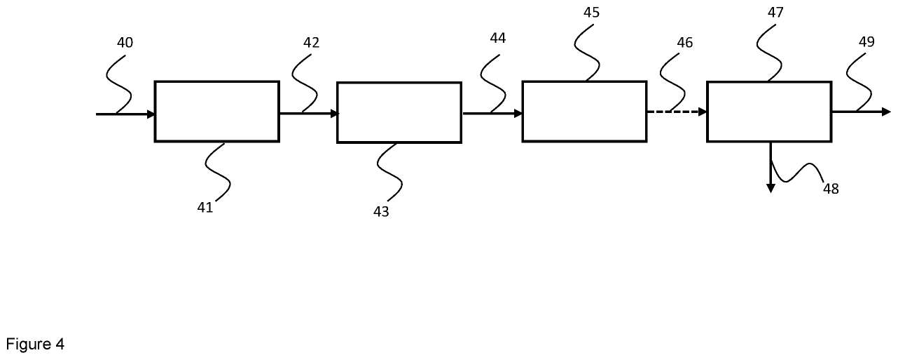

The system and method according to the present invention can be improved in a third embodiment by increasing the amount of H₂ in the clean syngas having a first molar ratio H₂:CO before entering the methanation unit which is shown on FIG. 4.

The system for converting a carbonaceous feedstock (40) into H₂ (49) and solid carbon (48) in a methane pyrolysis unit (47) according to the third embodiment of the present invention comprises
at least one syngas producing unit (41),
a methanation unit (45) and
a methane pyrolysis unit (47),
wherein the methanation producing unit (45) is downstream of and fluidically connected to said at least one syngas producing unit (41) and wherein said methane pyrolysis unit (47) is downstream of and fluidically connected to said methanation unit (45) or said methane pyrolysis unit (47) is downstream of and connected by a natural gas pipeline grid to said methanation unit (45),
wherein said system further comprises at least one syngas upgrading unit (43) which is downstream of and fluidically connected to the at least one syngas producing unit (41) or the at least one syngas purification unit of a gasification island and upstream of and fluidically connected to a methanation unit (45).

Preferably, said syngas producing unit (41) is a gasification island and wherein said gasification island comprising
at least one feedstock pre-treatment unit
at least one gasifier and
at least one syngas purification unit
wherein the at least one gasifier is downstream of and fluidically connected to the at least one feedstock pre-treatment unit and the at least one syngas purification unit is downstream of and fluidically connected to the at least one gasifier.

The H₂ concentration of the clean syngas (42) having a first molar ratio H₂:CO is increased in the syngas upgrading unit (43) which leads to a syngas (44) having a second molar ratio H₂:CO . Said second molar ratio H₂:CO is higher than the first molar ratio H₂:CO of the clean syngas (42). The at least one syngas upgrading unit (43) can be for example at least one water-gas shift unit or a H₂ injection unit which are both suited to increase the H₂ concentration in the clean syngas (42) having a first molar ratio H₂:CO to obtain the syngas (44) having a second molar ratio H₂:CO.

The method according to this third embodiment of the present invention further comprises between step (iii) and step (iv) the step (iiia): increasing the H₂ content in the clean syngas (42) having a first molar ratio H₂:CO to obtain a second syngas (44) having a second molar ratio H₂:CO which is higher than the first molar ratio H₂:CO of said clean syngas (42).

The disclosure in respect to the methanation unit (45) and the methane pyrolysis unit (47) made in respect to the first and second embodiments of the present invention applies also to the third embodiment. Accordingly, a stream comprising methane (46) is obtained in the methanation unit (45), said stream comprising methane (46) is then fed into a methane pyrolysis unit (47) downstream of and fluidically connected to the methanation unit (45) or transferred to a natural gas pipeline grid and transported in the natural gas pipeline grid to a second location and then inserted to a methane pyrolysis unit (47) or is temporarily stored in a suitable means for storage and then inserted to a methane pyrolysis unit (47). In both cases, the methane (46) is converted in the methane pyrolysis unit (47) into H₂ (49) and solid carbon (48). The first location where the at least one syngas producing unit (41), the at least one syngas upgrading unit (43) and the methanation unit (45) are placed and second location where the methane pyrolysis unit (47) is placed are different from each other in case the methane (46) is transported in a natural gas pipeline grid from the methanation unit (45) to the methane pyrolysis unit (47).

The third embodiment of the present invention can also be formulated as: subjecting a methane stream to methane pyrolysis, wherein the methane stream is derived from a process comprising
(i) conversion of a solid and/or liquid carbonaceous feedstock, preferably by gasification to obtain raw syngas,
(ii) removing impurities from said syngas to obtain a clean syngas, said clean syngas having a first molar ratio H₂:CO,
(iia) increasing the H₂ concentration in the clean syngas having a first molar ratio H₂:CO to obtain the syngas having a second molar ratio H₂:CO and
(iii) methanation of said clean syngas to obtain said methane stream.

The advantage of a second syngas (44) having said second molar ratio H₂:CO which is higher than the first molar ratio H₂:CO in the clean syngas (42) is a higher yield in the methanation reaction according to chemical reaction scheme (1).

A higher methane yield in the methanation unit (45) results in a higher H₂ and solid carbon yield in the methane pyrolysis reaction in respect to the amount of carbonaceous feedstock used in the gasification reaction. Hence, a higher yield of the desired reaction products H₂ and solid carbon and less of the undesired reaction product CO₂ are obtained by the third embodiment of the system and method according to the present invention.

Said at least one syngas upgrading unit (43) optionally further comprises a CO₂ capture unit downstream of and fluidically connected to the at least one syngas upgrading unit (43) and upstream of and fluidically connected to the methanation unit (45).

A CO₂ capture unit is also optionally used in the method according to the third embodiment of the present invention which then optionally comprises an additional step (iiib) between steps (iiia) and (iv) wherein CO₂ is removed from said second syngas (44) in said CO₂ capture unit. Hence, even less CO₂ is released into the atmosphere in this embodiment of the present invention. The CO₂ optionally captured can be stored and/or utilized e.g., for the synthesis of methanol.

This embodiment of the present invention can also be formulated as: subjecting a methane stream to methane pyrolysis, wherein the methane stream is derived from a process comprising
(i) conversion of a solid and/or liquid carbonaceous feedstock, preferably by gasification to obtain raw syngas,
(ii) removing impurities from said syngas to obtain a clean syngas, said clean syngas having a first molar ratio H₂:CO,
(iia) increasing the H₂ concentration in the clean syngas having a first molar ratio H₂:CO to obtain the syngas having a second molar ratio H₂:CO,
(iib) removing CO₂ from said syngas having a second molar ratio H₂:CO and
(iii) methanation of said clean syngas to obtain said methane stream.

A large variety of CO₂ capture units and methods for CO₂ capture are commercially used and can be selected and adapted by a skilled person to the systems and methods according to the present invention. Suitable methods for CO₂ removal from syngas include membrane separation, absorption and adsorption with e.g., pressure-swing-adsorbtion (PSA) or MOFs (metal organic frameworks).

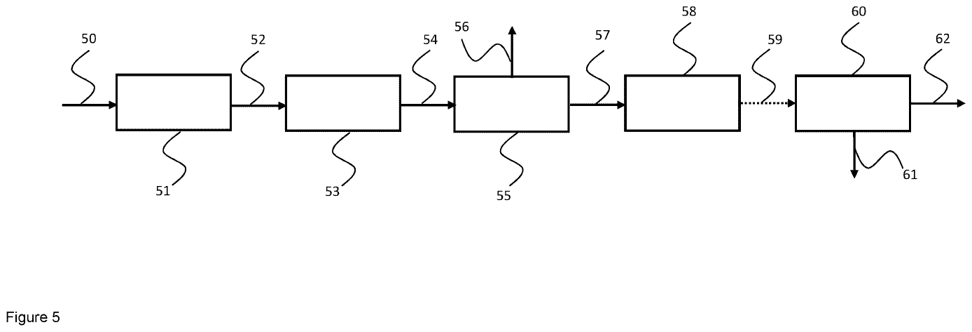

In a fourth embodiment of the present invention (FIG. 5), the clean syngas (52) having a first molar ratio H₂:CO is converted into a second syngas (54) having a second molar ratio H₂:CO in at least one syngas upgrader (53), wherein the H₂ content in said second molar ratio H₂:CO is higher than in said first molar ratio H₂:CO. The at least one syngas upgrader (53) in this fourth embodiment of the present invention is at least one water-gas shift unit.

The system and method according to the fourth embodiment comprises a syngas producing unit (51) which is preferably a gasification island comprising at least one gasifier. Said syngas producing unit (51) is upstream of and fluidically connected to at least one water-gas shift unit (53). The carbonaceous feedstock (50) enters the at least one syngas producing unit (51), clean syngas (52) having a first molar ratio H₂:CO leaves said at least one syngas producing unit (51) and enters said at least one water-gas shift unit (53). Said at least one syngas producing unit (51) preferably has the same components and properties as the at least one syngas producing unit (21) (FIG. 2) and/or the gasification island (FIG. 3, (31), (32), (33)).

The clean syngas (52) having a first molar ratio H₂:CO is then subjected to a water-gas shift reaction in the at least one water-gas shift unit (53). Thereby, the H₂ content in the clean syngas (52) is increased by reacting a portion of the CO of the clean syngas (52) with water to form additional H₂ (and CO₂) and thereby the second syngas (54) having a second molar ratio H₂:CO is formed and leaves the at least one water-gas shift unit (53). The H₂ content in said second syngas having a second molar ratio H₂:CO (54) is higher than in said clean syngas (52) having a first molar ratio H₂:CO. This step is known as water-gas shift reaction and represented by the chemical reaction scheme (4):

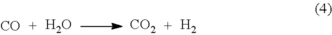

The water-gas shift reaction will operate with a variety of catalysts (such as copper-zinc-aluminum catalysts and chromium or copper promoted iron-based catalysts) in the temperature range between about 200° C. and about 480° C. The type of water-gas shift reaction und unit(s) required can be adapted to the general conditions of the process (e.g., type of carbonaceous feedstock and how much additional H₂ obtained by chemical reaction scheme (4) is desired).

The at least one water-gas shift unit (53) is upstream of and fluidically connected to a CO₂ capture unit (55). Said CO₂ capture unit (55) is upstream of and fluidically connected to the methanation unit (58). CO₂ (56) present in the second syngas (54) having a second molar ratio H₂:CO is removed from said second syngas (54) in the CO₂ capture unit (55) and a second syngas having a second molar ratio H₂:CO and in addition comprising a reduced amount of or almost no CO₂ (57) is leaving the CO₂ capture unit (55).

Next, CO₂ is removed from said second syngas (54) having a second molar ratio H₂:CO before said second syngas (54) enters the methanation unit (58). The methane (59) formed in the methanation unit (58) is then entering the methane pyrolysis unit (60) from which a H₂ stream (62) and solid carbon (61) leave.

Suitable methods for CO₂ removal from syngas include membrane separation, absorption and adsorption with e.g., pressure-swing-adsorption (PSA) MOFs (metal organic frameworks). Preferably, CO₂ is removed from said second syngas (54) having a second molar ratio H₂:CO by absorption: the second syngas (54) is contacted with an aqueous solution of alkylamines such as monoethanolamine, diethanolamine, methyldiethanolamine and the like and CO₂ (56) is captured in such aqueous solutions of alkylamines in an acid-base reaction. Said aqueous solution of alkylamines is then directed to a “regenerator” (e.g., a stripper with a boiler) where the acid-base reaction is reversed and whereby CO₂ and the recycled aqueous solution of alkylamines are obtained. This absorption method is also known as “scrubbing”. Commercially available scrubbing technologies suitable for the systems and methods according to the present invention are for example marketed under the brand name OASE® which are available from BASF SE.

The system according to the fourth embodiment of the present invention further comprises a methanation unit (58) and a methane pyrolysis unit (59). The CO₂ capture unit (55) is upstream of and fluidically connected to the methanation unit (58). Said methane pyrolysis unit (59) is downstream of and fluidically connected to the methanation unit (58) or the methane pyrolysis unit (60) is downstream of and connected by a natural gas pipeline grid to the methanation unit (58).

The system according to the present invention is also suitable for the following method: Subjecting a methane stream to methane pyrolysis, wherein the methane stream is derived from a process comprising
(i) conversion of a solid and/or liquid carbonaceous feedstock, preferably by gasification to obtain raw syngas,
(ii) removing impurities from said syngas to obtain a clean syngas, said clean syngas having a first molar ratio H₂:CO,
(iia) increasing the H₂ concentration in the clean syngas having a first molar ratio H₂:CO to obtain the syngas having a second molar ratio H₂:CO by a water-gas shift reaction,
(iib) removing the CO₂ from said syngas having a second molar ratio H₂:CO and
(iii) methanation of said syngas having a second molar ratio H₂:CO to obtain said methane stream.

The disclosure in respect to said methanation unit (58) and said methane pyrolysis unit (60) made in respect to all other embodiments of the present invention applies also to the fourth embodiment of the present invention. Accordingly, a stream comprising methane (59) is obtained in the methanation unit (58), said stream comprising methane (59) is then fed into a methane pyrolysis unit (60). A H₂ stream (62) and solid carbon (61) are obtained from the methane pyrolysis reaction in the methane pyrolysis unit (60).

The advantage of a system and method according to the fourth embodiment are the same as those disclosed above for the third embodiment. Hence, a higher yield of methane (59) and likewise a higher yield of H₂ (62) and solid carbon (61) are obtained from the second syngas (54) having a second molar ratio H₂:CO which is higher than the first molar ration of the syngas (52) having a first molar ratio H₂:CO. Furthermore, the CO₂ formed in the syngas producing unit (51) is removed by conversion into solid carbon (61) and those portions of the CO₂ which are generated by the water-gas shift reaction are captured in the CO₂ capture (CC) unit (55) and are stored (CCS) and/or further utilized (CCU) and are, accordingly, not emitted into the atmosphere.

This fourth embodiment of the present invention is particularly preferred in case the carbonaceous feedstock comprises a high amount of biomass and therefore the CO₂ generated by converting the syngas obtained from such a carbonaceous feedstock in a water-gas shift unit has a high content of biogenic carbon. Additionally, when compared to the fifth embodiment of the present invention, no energy from renewable sources which is may be scarce is required in the fourth embodiment of the present invention to increase the H₂ content in the syngas.

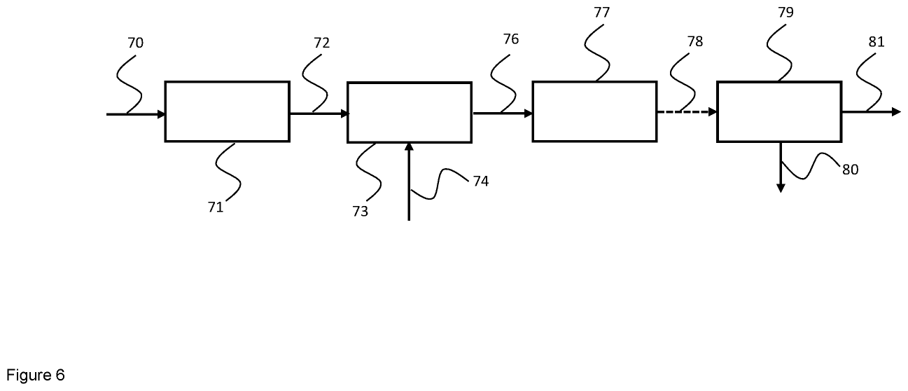

The system and method according to the fifth embodiment (FIG. 6) comprises at least one syngas producing unit (71) which is preferably a gasification island comprising at least one gasifier. Said at least one syngas producing unit (71) preferably has the same components and properties as the syngas producing unit (21) (FIG. 2) and/or the gasification island (FIG. 3, (31), (32), (33)) described above.

Said at least one syngas producing unit (71) is upstream of and fluidically connected to a H₂ insertion unit (73). The carbonaceous feedstock (70) enters the at least one syngas producing unit (71), clean syngas (72) having a first molar ratio H₂:CO leaves said at least one syngas producing unit (71) and enters said H₂ insertion unit (73).

The clean syngas (72) having a first molar ratio H₂:CO is converted into the second syngas (76) having a second molar ratio H₂:CO in step (iiia) by adding a suitable amount of additional H₂ (74) to the clean syngas (72) having a first molar ratio H₂:CO in the H₂ insertion unit (73). The additional H₂ (74) is preferably “green H₂” formed by water electrolysis using electricity generated from a renewable energy source. Suitable renewable energy sources comprise solar energy, wind energy, geothermal energy, hydropower, ocean energy and the like.

“Suitable amount of additional H₂” is defined herein as the amount of H₂ required to obtain the second syngas (75) having a second molar ratio H₂:CO of about 3:1 to about 4:1, most preferably of about 3:1 from the clean first syngas (72) having a first molar ratio H₂:CO of about 0.7:1 to about 1.1:1. The skilled person knows how to calculate said suitable amount of H₂ from the molar ratio H₂:CO and e.g., the flow rate of H₂ and CO in the clean syngas (72) having a first molar ratio H₂:CO and the desired second molar ratio H₂:CO of the second syngas (75). The H₂ insertion unit (73) can be for example a tee-piece connector with an optional static mixer.

The system according to the fifth embodiment of the present invention further comprises a methanation unit (77) and a methane pyrolysis unit (79). The H₂ insertion unit (73) is upstream of and fluidically connected to the methanation unit (77). The methane pyrolysis unit (79) is upstream of and fluidically connected to the methanation unit (77) or the methane pyrolysis unit (79) is downstream of and connected by a natural gas pipeline grid to the methanation unit (77). The methane (78) is then converted into H₂ (81) and solid carbon (80) in the methane pyrolysis unit (79). The first location where the at least one syngas producing unit (71), the H₂ insertion unit (73) and the methanation unit (77) are installed and the second location where the methane pyrolysis unit (79) is installed are different from each other in case the methane (78) is transported by a natural gas pipeline grid from the methanation unit (77) to the methane pyrolysis unit (79).

The disclosure in respect to said methanation unit (77) and said methane pyrolysis unit (79) made in respect to all other embodiments of the present invention applies also to the fifth embodiment. Accordingly, a stream comprising methane (78) is obtained in the methanation unit (77), said stream comprising methane (78) is then fed into a methane pyrolysis unit (79). A H₂ stream (81) and solid carbon (80) are obtained from the methane pyrolysis reaction in the methane pyrolysis unit (79).

The advantage of a system and method according to the fifth embodiment are the same as those disclosed above for the third and fourth embodiments. Hence, a higher yield of methane (78) and likewise a higher yield of H₂ (81) and solid carbon (80) are obtained from the second syngas (76) having a second molar ratio H₂:CO which is higher than the first molar ratio of the syngas (72) having a first molar ratio H₂:CO. Furthermore, the CO₂ formed in the syngas producing unit (71) is removed by conversion into solid carbon (80).

The system according to the present invention is also suitable for the following method: Subjecting a methane stream to methane pyrolysis, wherein the methane stream is derived from a process comprising
(i) conversion of a solid and/or liquid carbonaceous feedstock, preferably by gasification to obtain raw syngas,
(ii) removing impurities from said syngas to obtain a clean syngas, said clean syngas having a first molar ratio H₂:CO,
(iia) increasing the H₂ concentration in the clean syngas having a first molar ratio H₂:CO to obtain the syngas having a second molar ratio H₂:CO by insertion of additional H₂ and
(iii) methanation of said clean syngas to obtain said methane stream.

The CO₂ formed in the syngas producing unit (71), preferably in a gasifier, more preferably, in a gasification island comprising at least one gasifier, will be converted to additional methane in the methanation unit (77) because of the additional H₂ (74) added to the clean first syngas (72). The reaction of the CO₂ formed in the syngas producing unit (71) with additional H₂ (74) in the methanation unit (77) is represented by chemical reaction scheme (2). Hence, the system according to the fifth embodiment of the present invention preferably does not comprise a CO₂ capture (CC) unit (55) such as the one in the system according to the fourth embodiment of the present invention because all CO₂ formed in the syngas producing unit (71) is converted to methane (78) in the methanation unit (77).

The fifth embodiment is particularly preferred in case “green H₂” formed by water electrolysis using electricity generated from a renewable energy source is used as additional H₂ (74) because the methane yield in the methanation reaction (chemical reaction scheme (1)) can be increased without forming additional CO₂ as in case of the fourth embodiment by the water-gas shift reaction.

The use of “green H₂” as additional H₂ (74) (i.e., H₂ obtained by water electrolysis using a renewable energy source) results in methane (78) having a lower product carbon footprint (PCF) compared to a) “not-green H₂” as additional H₂ (74) and b) using a water-gas shift reaction combined with CO₂ capture (CC) to increase the methane yield in the methanation unit (77) instead of additional H₂ (74).

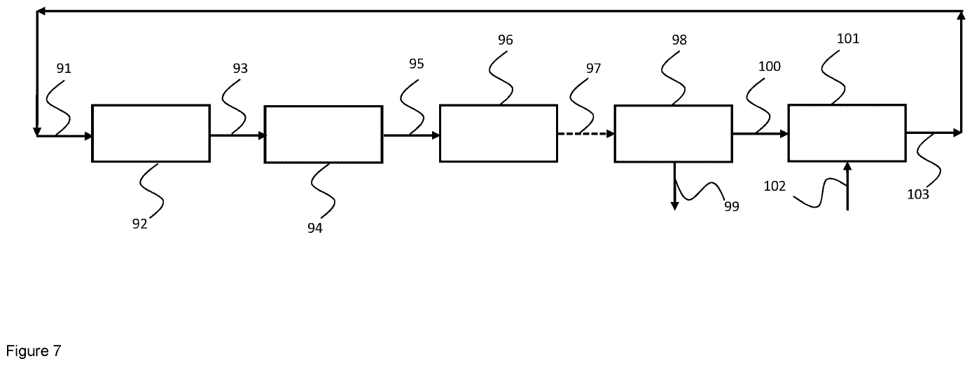

The system and the method according to the present invention and all their respective embodiments are suitable for implementation in a closed recycling loop for at least one chemical product. Such a closed recycling loop is shown in FIG. 7: at least one chemical product (103) is manufactured in at least one chemical reactor (101) using H₂ (100) obtained from the methane pyrolysis unit (98) and at least one second feedstock (102) containing one or more carbon atom. The at least one chemical product (103) becomes a carbonaceous feedstock (91) after it is disposed. Said carbonaceous feedstock (91) is then converted into a clean syngas (93) having a first molar ratio H₂:CO in at least one syngas producing unit (92) which is preferably a gasification island comprising at least one gasifier. Said clean syngas (93) having a first molar ratio H₂:CO is then converted in a methanation unit (96) into methane (97). Next, methane (97) is fed into a methane pyrolysis unit (98) which is downstream of and physically connected to the methanation unit (96) or the methane (97) is transferred to a natural gas pipeline and transported in the natural gas pipeline from the methanation unit (96) to a second location and there inserted into a methane pyrolysis unit (98) in which the methane (97) is converted into H₂ (100) and solid carbon (101). The methane pyrolysis unit (98) is downstream of the methanation unit (96). The first location where the at least one syngas producing unit (92) and the methanation unit (96) are installed and second location where the methane pyrolysis unit (98) is installed are different from each other in case the methane (97) is transported from the methanation unit (96) to the methane pyrolysis unit (98) in a natural gas pipeline grid.

The H₂ (100) obtained in the methane pyrolysis unit (98) is then used as a feedstock for manufacturing at least one chemical product in at least one chemical reactor (101). For example, H₂ (100) is used as a feedstock together with CO₂ as a second feedstock (102) for producing methanol which is then used as a feedstock for further conversions, including multistep conversions, into e.g., olefines and olefines into organic polymers such as polyethylene. An end-user product such as a plastic bag is then made from polyethylene and said plastic bag becomes (part of) a carbonaceous feedstock (91) after it is disposed as e.g., municipal solid waste (MSW).

This embodiment can be used in a method for converting a carbonaceous feedstock into H₂ and solid carbon comprising, in this order, the steps
(i) providing a carbonaceous feedstock from waste comprising a chemical product,
(ii) converting said carbonaceous feedstock into syngas,
(iii) removing impurities from said syngas to obtain a clean syngas, said clean syngas having a first molar ratio H₂:CO,
(iv) converting said clean syngas having a first molar ratio H₂:CO with a methanation reaction into methane,
(v) converting said methane with a methane pyrolysis reaction into H₂ and solid carbon,
(vi) forming said chemical product comprised in the said carbonaceous feedstock directly or indirectly from said H₂ obtained in step (v) and at least one second feedstock containing one or more carbon atom.

Optionally, the clean syngas (93) having a first molar ratio H₂:CO is converted in at least one syngas upgrading unit (94) into a second syngas (95) having a second molar ratio H₂:CO wherein said second molar ratio H₂:CO is larger than said first molar ratio H₂:CO. Said second syngas (95) having a second molar ratio H₂:CO is then converted in a methanation unit (96) into methane (97). The advantages of such a conversion of syngas are already described above for the third, fourth and fifth embodiment and apply also to this embodiment of the present invention. The at least one syngas upgrading unit (94) can be for example at least one water-gas shift unit (preferably with a successive CO₂ capture unit downstream of and fluidically connected to said at least one water-gas shift unit) or a H₂ injection unit. Such optional and additional unit operations and their implementation into the system and method according to the present invention are described above. The same variants and advantages also apply to the closed recycling loop embodiment described here and shown in FIG. 7.

1.-15. (canceled)

16. A system for converting a carbonaceous feedstock into H₂ and solid carbon, the system comprising:
at least one syngas producing unit,
a methanation unit,
a methane pyrolysis unit and
wherein the methanation unit is downstream of and fluidically connected to the syngas producing unit and
wherein the methane pyrolysis unit is downstream of and fluidically connected to the methanation unit or the methane pyrolysis unit is downstream of and connected by a natural gas pipeline grid to the methanation unit.

17. The system for converting a carbonaceous feedstock into H₂ and solid carbon according to any of claim 16, wherein the at least one syngas producing unit is a gasification island, said gasification island comprising:
at least one feedstock pre-treatment unit
at least one gasifier,
at least one syngas purification unit and
wherein the at least one gasifier is downstream of and fluidically connected to the at least one feedstock pre-treatment unit and the at least one syngas purification unit is downstream of and fluidically connected to the at least one gasifier.

18. The system for converting a carbonaceous feedstock into H₂ and solid carbon according to claim 16, wherein the system further comprises at least one syngas upgrading unit which is downstream of and fluidically connected to the at least one syngas producing unit or the at least one syngas purification unit of a gasification island and upstream of and fluidically connected to a methanation unit.

19. The system for converting a carbonaceous feedstock into H₂ and solid carbon according to claim 18, wherein the at least one syngas upgrading unit comprises in this order and fluidically connected to each other, at least one water-gas shift unit and at least one CO₂ capture unit, wherein said at least one water-gas shift unit is downstream of and fluidically connected to the at least one syngas producing unit and/or the at least one syngas purification unit of a gasification island and wherein the at least one CO₂ capture unit is upstream of and fluidically connected to said methanation unit.

20. The system for converting a carbonaceous feedstock into H₂ and solid carbon according to claim 16, wherein the at least one syngas upgrading unit comprises a H₂ injection unit which is downstream of and fluidically connected to said at least one syngas producing unit and/or the at least one syngas purification unit of a gasification island and wherein said H₂ injection unit is upstream of and fluidically connected to said methanation unit.

21. The system according to claim 16, wherein the methane pyrolysis unit is downstream of and connected by a natural gas pipeline grid to the methanation unit.

22. The system according to claim 16, wherein the methane pyrolysis unit is downstream of and fluidically connected to the methanation unit.

23. A method for converting a carbonaceous feedstock into H₂ and solid carbon comprising, in this order, the steps:
(i) providing a carbonaceous feedstock, optionally from waste comprising a chemical product,
(ii) converting said carbonaceous feedstock into syngas,
(iii) removing impurities from said syngas to obtain a clean syngas, said clean syngas having a first molar ratio H₂:CO,
(iv) converting said clean syngas having a first molar ratio H₂:CO with a methanation reaction into methane, and
(v) converting the methane with a methane pyrolysis reaction into H₂ and solid carbon, optionally forming said chemical product comprised in the said carbonaceous feedstock directly or indirectly from the H₂ obtained in step (v) and at least one second feedstock containing one or more carbon atom.

24. The method for converting a carbonaceous feedstock into H₂ and solid carbon according to claim 23, wherein said carbonaceous feedstock is converted into syngas by a gasification reaction and/or wherein the carbonaceous feedstock is selected from the group comprising selected from the group comprising biomass, municipal solid waste (MSW), shredder residues such as car shredder residues, textiles and mixtures of the aforementioned.

25. The method for converting a carbonaceous feedstock into H₂ and solid carbon according to claim 23, wherein the method comprises a further step between step (iii) and step (iv):
(iiia) converting said clean syngas having a first molar ratio H₂:CO into a second syngas, said second syngas having a second molar ratio H₂:CO
wherein the H₂ content in said second molar ratio H₂:CO is higher than in said first molar ratio H₂:CO.

26. The method for converting a carbonaceous feedstock into H₂ and solid carbon according to claim 25, wherein the method comprises a further step between step (iiia) and step (iv): (iiib) removing CO₂ from said second syngas having a second molar ratio H₂:CO.

27. The method for converting a carbonaceous feedstock into H₂ and solid carbon according to claim 25, wherein said clean syngas having a first molar ratio H₂:CO formed in step (iii) is converted into said second syngas having a second molar ratio H₂:CO in step (iiia) by a watergas shift reaction.

28. The method for converting a carbonaceous feedstock into H₂ and solid carbon according to claim 25, wherein said clean syngas having a first molar ratio H₂:CO formed in step (iii) is converted into said second syngas having a second molar ratio H₂:CO in step (iiia) by insertion of additional H₂ into said clean syngas having a first molar ratio H₂:CO, optionally, said H₂ is formed by water electrolysis using electricity generated from a renewable energy source.

29. The method for converting a carbonaceous feedstock into H₂ and solid carbon according to claim 23, wherein the methane formed in step (iv) is transported through a natural gas pipeline grid in an additional step (iv′) prior to converting the methane with a methane pyrolysis reaction into H₂ and solid carbon in step (v) and wherein steps (i) to (iv) are carried out in a first location and step (v) is carried out in a second location and wherein the first location and the second location are different from each other.

30. A method of deriving a methane stream configured for use in methane pyrolysis, the method comprising:
(i) gasification of a solid and/or liquid carbonaceous feedstock to obtain syngas,
(ii) removing impurities from said syngas to obtain a clean syngas, said clean syngas having a first molar ratio H₂:CO, and
(iii) methanation of said clean syngas to obtain the methane stream.
